(19) United States
(12) Patent Application Publication
(10) Pub. No.: US 2007/0292522 A1
(43) Pub. Date: Dec. 20, 2007

(54) Method for forming microcapsules with multiple shell layers

(76) Inventors: Jeh-Yi Chiu, Taipei (TW); Choh-Yu Chen, Taipei (TW); Ching-Hoa Huang, Taipei (TW)

Correspondence Address: JEH-YI CHIU;235 Chung-Ho, Box 8-24, Taipei, R.O.C., omitted

(21) Appl. No.: 11/541,594

(22) Filed: Oct. 3, 2006

(30) Foreign Application Priority Data
Jun. 20, 2006 (TW) 095122169

(51) Int. Cl. A61K 9/50; A61K 9/16

(52) U.S. Cl. 424/490; 264/4.1; 977/906

(57) ABSTRACT

A method for forming microcapsules comprises the steps of: forming a core; enclosing a first, a second and a third layer of shells sequentially; wherein the microcapsules has one of the three properties of anti-ultraviolet and can endure a high temperature of 280° C.; and temperature sensitive and color changed and can endure a high temperature of 280° C., and endure a high temperature of 280° C. Furthermore, the three layers of the shells encloses the core so that the releasing speed of the material or drug therein is changeable, and the tension of the micro capsule is changed so as to adjust the size of the microcapsules to be between 0.01˜1000 Micron.

### FIELD OF THE INVENTION

The present invention relates to microcapsules, and in particular to a method for manufacturing microcapsules which contain a core and three shells. The arrangement of different shells with different properties causes that the microcapsules provide various functions.

### BACKGROUND OF THE INVENTION

The conventional way for manufacturing microcapsules is mainly by interface polymerization. This method has many advantages, such as quick reaction, purity of the monomers is low and the ratio of the monomers is not strictly confined. However the prior art microcapsule is not a temperature endurable one and the properties thereof is not steady, such as it is difficult to have the properties of color change of high ultraviolet sensitivity and high temperature endurance of 280° C., or color change of temperature sensitivity and high temperature endurance of 280° C. The prior art has many improvements for achieving the basic requirement, such as those disclosed in U.S. Pat. No. 5,281,570 by various combinations of the chemical prescriptions. However all these improvements cannot achieve desired properties and thus they are necessary to be improved.

### SUMMARY OF THE INVENTION

To improve the defect in the prior art, the present invention provides a method for forming microcapsules which comprises the steps of: forming a core; enclosing a first, a second and a third layer of shells sequentially; wherein the microcapsules have one of the three properties of (1) anti-ultraviolet and endurance of high temperature of 280° C.; and (2) being temperature sensitive and color changeable and endurance of high temperature of 280° C., and (3) endurance of high temperature of 280° C. Furthermore, the three layers of the shells enclose the core so that the releasing speed of the material or drug therein is changeable, and the tension of the micro capsule is changed to adjust the size of the microcapsules to be between 0.01˜1000 Micron.

### DETAILED DESCRIPTION OF THE INVENTION

In order that those skilled in the art can further understand the present invention, a description will be described in the following in details. However, these descriptions and the appended drawings are only used to cause those skilled in the art to understand the objects, features, and characteristics of the present invention, but not to be used to confine the scope and spirit of the present invention defined in the appended claims.

### First Embodiment

10% surfactant is added with de-ionized water as a solution. Then the mixture is mixed with 30% triethanolamine in a container. The PH value (Pondus Hydrogenii value for determining acidity and alkalinity of a solution) is controlled to be above 4-5 and the temperature is increased to be between 70 to 80° C. Then the 100 grams of ethylstearate is added to the solution. Then the solution is agitated by a homo-emulsifier with a speed of 7000-8000 rpm through a time period over one minute. Then 40%, 12 to 15 grams of Melamine-formaldehyde resin as a first shell material is added thereto and then is agitated through 1 to 3 minutes with a speed of 7500 rpm. Then some samples are taken for checking the diameter of particles. Then the second shell is performed. The material of the second shell is high molecular melamine derivatives with a weight of 18 to 20 grams and a rotation speed of 7500 rpm. The reaction is performed through 3 minutes. Then some samples are taken for checking the diameter of particles. Then the enclosing of the third shell is performed. The material of the third shell is titanium coupling agent and cross-linker with a ratio of 2:1. Then the product is mixed and agitated with a rotation speed of 8000 rpm through 3 minutes. When it is assured that the third shell is enclosed, the agitate speed is reduced to 400 rpm under a temperature of 80 to 90° C. through 3 hours. After 3 hours, 50% citric acid, 18 grams of melamine-formaldehyde resin, and 200 grams of de-ionized water are added sequentially and the product is agitated under a speed of 400 rpm through several minutes for getting the microcapsules of particle diameters of 8 to 16 μm.

### Second Embodiment

10% surfactant is added with de-ionized water. Then the mixing solution is mixed with 30% triethanolamine in a container. The PH value (Pondus Hydrogenii value for determining acidity and alkalinity of a solution) is controlled to be above 4-5 and the temperature is increased to be between 70 to 80° C. The mixing material is prepared. In that, 100 grams of 3-2-Ethy Hexanoic Acid glyceride, 12 grams of light sensitive color changed powders and 18 grams of ultraviolet absorbers are mixed and then the mixing material is added to the container with triethanolamine. Then the solution is agitated by a homo-emulsifier with a speed of 7000-8000 rpm through above one minute so as to form cores of microcapsules. Then 12 to 15 grams of Melamine-formaldehyde resin as a first shell material is added thereto is added and agitated through 1 to 3 minutes with a speed of 7500 rpm. Then some samples are taken for checking the diameter of particles. Then the enclosing of the second shell is performed. The material of the second shell is high molecular melamine derivatives with a weight of 18 to 20 grams under a rotation speed of 7500 rpm through 3 minutes. Then some samples are taken for checking the diameter of particles. Then the enclosing of the third shell is performed. The material of the third shell is titanium coupling agent and cross-linker with a ratio of 2:1. Then the product is mixed and agitated with a rotation speed of 8000 rpm through 3 minutes. When it is assured that the third shell is enclosed, the agitate speed is reduced to 400 rpm under a temperature of 80 to 90° C. through 3 hours. After 3 hours, 50% citric acid, 18 grams of melamine-formaldehyde resin, and 200 grams of de-ionized water sequentially and the product is agitated under a speed of 400 rpm through several minutes for getting the microcapsules of particle diameters of 8 to 16 μm which is anti-ultraviolet and can endure a high temperature of 280° C. through one hour.

### Third Embodiment

10% surfactant is added with de-ionized water. Then the mixing solution is mixed with 30% triethanolamine in a container. The PH value (Pondus Hydrogenii value for determining acidity and alkalinity of a solution) is controlled to be above 4-5 and the temperature is increased to be between 70 to 80° C. The mixing material is prepared. In that, 76 grams of ethylstearate, 8 grams of crystallized lactone CVL, 16 grams of pigments, and 1 to 2 grams of oil-soluable heat stabilizers, ultraviolet absorber, and light stabilizer are added to the containers. Then the solution is agitated by a homo-emulsifier with a speed of 7000-8000 rpm through above one minute so as to form cores of microcapsules. Then 12 to 15 grams of Melamine-formaldehyde resin as a first shell material is added thereto is added and agitated through 1 to 3 minutes with a speed of 7500 rpm. Then some samples are taken for checking the diameter of particles. Then the enclosing the second shell is performed. The material of the second shell is high molecular melamine derivatives with a weight of 18 to 20 grams with a rotation speed of 7500 rpm through 3 minutes. Then some samples are taken for checking the diameter of particles. Then the enclosing the third shell is performed. The material of the third shell is titanium coupling agent and cross-linker with a ratio of 2:1. Then the product is mixed and agitated with a rotation speed of 8600 rpm through 3 minutes. When it is assured that the third shell is enclosed, the agitate speed is reduced to 400 rpm under a temperature of 80 to 90° C. through 3 hours. After 3 hours, 50% citric acid, 18 grams of melamine-formaldehyde resin, and 200 grams of de-ionized water sequentially and the product is agitated under a speed of 400 rpm through several minutes for getting the microcapsules of particle diameters of 8 to 16 μm which is anti-ultraviolet and can endure a high temperature of 280° C. through one hour.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for forming microcapsules comprising the steps of:
forming a core;
enclosing a first, a second and a third layer of shells sequentially;
whereby the microcapsules has one of the three properties of: anti-ultraviolet and can endure a high temperature of 280° C.; and temperature sensitive and color changed and can endure a high temperature of 280° C.; and endure a high temperature of 280° C.

2. A method for forming microcapsules comprising the steps of:
forming a core;
enclosing a first, a second and a third layer of shells sequentially;
wherein the three layers of the shells encloses the core so that the releasing speed of the material or drug therein is changeable, and the tension of the micro capsule is changed so as to adjust the size of the microcapsules to be between 0.01˜1000 Micron.

3. The method of claim 1, wherein the micro capsule is oil solvable material mixed with oil solvable dyes, or oil solvable hot stabilizer or oil solvable light stabilizer, or oil solvable ultraviolet absorber, or surfactant, the oil solvable material is selected from essential oil, white wax oil, fatty acid, carbon alcohol, ester, and ketone.

4. The method of claim 1, wherein the first shell is water solvable high molecular compound material selected from melamine formaldehyde resin, acrylic resin, gelatin, urea formaldehyde resin, and polyvinyl alcohol resin.

5. The method of claim 1, wherein a material of the second layer is selected from hydrophile coupling agent and cross-linker.

6. The method of claim 1, wherein the third layer is elected from hydrophile coupling agent and nano-material anti-bacteria agent.

7. The method of claim 1, wherein the third layer contains the mixing material of zinc oxide, 3-2-Ethy Hexanoic Acid glyceride and silver ions.

8. The method of claim 1, wherein the core is formed by adding 10% surfactant with de-ionized water; then the mixing solution being mixed with 30% triethanolamine in a container; the PH value being controlled to be above 4-5 and increasing the temperature to be between 70 to 80° C.; then adding 100 grams of ethylstearate to the solution; then agitating the solution by a homo-emulsifier with a speed of 7000-8000 rpm through one to three minutes; then 40%, 12 to 15 grams of Melamine-formaldehyde resin as a first shell material is added thereto is added and agitated through 1 to 3 minutes with a speed of 7500 rmp; and then samples are taken for checking the diameter of particles.

9. The method of claim 5, wherein the rotation speed of manufacturing of the third shell is controlled to be between 7000 to 8500 rpm through one to five minutes, then agitating with a rotation speed of 400 rpm, and the operation temperature is retained at 80° C. through three hours.
